(19) United States
(12) Patent Application Publication
(10) Pub. No.: US 2005/0080109 A1
(43) Pub. Date: Apr. 14, 2005

(54) Gamma-tocopherol and gamma-tocotrienol therapy for multiple sclerosis

(76) Inventors: Andreas M. Papas, Kingsport, TN (US); Konstantinos A. Papas, Jonesborough, TN (US)

Correspondence Address: SUSAN F. JOHNSTON;SUSAN F. JOHNSTON, ATTORNEY AT LAW, P.O. BOX 4449, JOHNSON CITY, TN, 37602-4449, US

(21) Appl. No.: 10/962,391

(22) Filed: Oct. 12, 2004

Related U.S. Application Data
(60) Provisional application No. 60/510,210, filed on Oct. 9, 2003.

(51) Int. Cl. A61K 31/355

(52) U.S. Cl. 514/337; 514/458

(57) ABSTRACT

A therapy for decreasing the rate of demyelination in multiple sclerosis comprising administering gamma-tocopherol and/or gamma-tocotrienol in a therapeutically effective dosage. The therapeutic dosage is high, between about 800 mg to about 10 grams of gamma-tocopherol and between about 50 mg to about 5 grams of gamma-tocotrienol per day.

### CROSS REFERENCES TO RELATED APPLICATIONS

This application claims the benefit of U.S. Provisional Application Ser. No. 60/510,210 filed Oct. 9, 2003.

### FIELD OF THE INVENTION

The present invention relates to methods for treating multiple sclerosis using antioxidants.

### BACKGROUND OF THE INVENTION

Multiple sclerosis (MS) is a progressive, inflammatory disease of the central nervous system (CNS). Many investigators believe MS to be an autoimmune disease in which inflammation occurs in the white matter of the CNS. MS lesions, characterized by perivascular infiltration of monocytes and lymphocytes, appear as indurated areas in pathologic specimens; hence, the term “sclerosis in plaques.” Continuation of this inflammation results in loss of the nerve-insulating myelin with preservation of the axons or fiber tracts, a process known as demyelination. In MS, demyelination occurs within the central nervous system and is referred to as central demyelination. Demyelination ultimately results in nervous system scars, called plaques, which interrupt communications between the nerves and the reset of the body. Magnetic resonance imaging (MRI) characteristically shows lesions of high T2 signal intensity of variable location in the white matter of the brain, brain stem, optic nerves, or spinal cord. Demyelination is the root cause of the symptoms that MS patients experience because it causes the speed at which messages pass along nerves to be slower than normal. Even when the patches of scarring caused by demyelination have healed and re-myelination has occurred, the response time of nerve endings tends to remain slower.

There is no known cure for MS. Current drug treatment for delaying the progression of the disease and reducing the number of new MS lesions include immunomodulators such as interferon beta-1a and interferon beta-1b. Interferon beta-1a and interferon beta-1b are believed to act via counteracting cell surface expression of pro-inflammatory or pro-adhesion molecules on immune cells, among other effects. Further, corticosteroids are used to reduce acute inflammation and expedite recovery from acute exacerbations of MS. They are used for “rescue” therapy as monthly boosters in patients who respond poorly to immunomodulators. Immunosuppressors are also used for their ability to suppress immune reactions in MS.

Thus, it is an object of the present invention to provide a means for reducing the amount of demyelination in MS and thus improving the physical state and quality of life for MS patients.

### SUMMARY OF THE INVENTION

The present invention includes a method for decreasing the rate of demyelination in multiple sclerosis. The method includes administering a compound selected from the group consisting of gamma-tocopherol, an ester of gamma-tocopherol, gamma-tocotrienol, an ester of gamma-tocotrienol, and a combination thereof in a therapeutically effective dosage for decreasing the rate of demyelination of the central nervous system of an MS patient. The therapeutic dosage of gamma-tocopherol and/or gamma-tocotrienol, in this invention, is a much higher amount than a dietary supplement dosage of the same nutrients.

The present invention further includes a medicament useful for reducing the rate of demyelination in multiple sclerosis. A unit dosage of the medicament includes a therapeutically effective amount of an agent selected from the group consisting of gamma-tocopherol, an ester of gamma-tocopherol, gamma-tocotrienol, an ester of gamma-tocotrienol, and a combination thereof.

### DETAILED DESCRIPTION OF THE INVENTION

The inventors have found that gamma-tocopherol, gamma-tocotrienol, and suitable esters thereof (referred to collectively herein as “gamma-T”) reduce inflammation and demyelination in MS when delivered in therapeutically effective doses. A poignant aspect of the present invention is that a therapeutically effective dose of gamma-T in the present invention is much higher than a dietary supplementary dose of gamma-T. The gamma-T therapy of the present invention reduces or stops the inflammatory cascade of the CNS which leads to demyelination, and thus, the various symptoms of demyelination such as trembling and slow response. Accordingly, the rate of demyelination is reduced, preferably being reduced to a rate of zero for no demyelination at all.

The present method for treating MS comprises administering gamma-tocopherol, gamma-tocotrienol, an ester thereof, or a combination thereof to an MS patient in a daily dosage effective for reducing demyelination. Decreased rate of demyelination is shown in tissue cultures via staining methods known well in the art. In clinical practice, a reduction in the rate of demyelination for purposes of the present invention is considered to be attained when known diagnostic methods such as MRI testing, and cerebral spinal fluid testing, over a period of time indicate less demyelination. Additionally, a reduction in the rate of demyelination is indicated by fewer episodes of acute exacerbations of MS and by the stabilization or improvement of symptoms and function.

The therapeutically effective dosage of gamma-T in the present invention is that amount that causes a reduction in demyelination or a reduction in the rate of demyelination. The daily dosage size of gamma-tocopherol or its esters in the present invention is between about 800 mg to about 10 grams, assuming an average body weight of about 70 kg, with a preferred daily dosage of greater than 1 gram to about 7.5 grams, and a more preferred daily dosage of gamma-tocopherol being between about 2 to 5 grams. This is far greater than a daily dietary supplement dose of gamma-tocopherol.

The daily dosage size of gamma-tocotrienol or its esters in the present invention is between about 50 mg to about 5 grams, preferably between about 100 mg to about 2 grams, with a more preferred dosage range being between about 200 mg to about 1.5 grams per day. Likewise, this is far greater than a daily dietary supplement dose of gamma-tocotrienol. Again, these dosage ranges are based on a patient population having an average body weight of about 70 kg.

The ratio of gamma-tocopherol to gamma-tocotrienol administered in the present invention is not critical and should be altered according to the particular MS disease state of the patient. Both gamma-tocopherol, administered alone, and gamma-tocotrienol, administered alone, show unexpected therapeutic effects in MS.

The present method is additionally beneficial when a therapeutically effective amount of coenzyme Q10 is administered with said gamma-T. A weight ratio of at least 2:1 gamma-T to coenzyme Q10 is preferred, with a weight ratio of about 2:1 to about 10:1 gamma-T to coenzyme Q10 being more preferred. Other nutrients particularly beneficial for administration along with gamma-T in this invention include omega-3 fatty acids, omega-6 fatty acids, alpha-lipoic acid, astaxanthin, alpha-, beta-, and delta-tocopherol, alpha-, beta-, and delta-tocotrienol, and the isoflavones including genistein, daidzein, glycitein, equol, formononetin, and biochanin.

The present method for treating MS is most beneficially conducted as a chronically administered therapy, preferably via oral administration of gamma-T. However, the method is also beneficially conducted as an acutely administered therapy during or after acute exacerbations of MS. An acute administration is beneficially delivered orally or intra-thecally, for example, into the tissue surrounding the spinal cord.

The present invention includes a medicament for reducing central demyelination in MS wherein a unit dosage thereof includes a therapeutically effective amount of gamma-tocopherol, gamma-tocotrienol, esters thereof, or a combination thereof. A suitable oral medicament for systemic treatment can be in the form of an oil, a gel capsule, a tablet, or an aqueous emulsion. A suitable injectable medicament would be an aqueous emulsion and can be used for systemic treatment or acute therapy.

Suitable esters of gamma-tocopherol, gamma-tocotrienols, as well as the other tocopherols and tocotrienols used herein include, the succinate esters, polyethylene glycol succinate esters, acetates, nicotinates, and phosphates. D-alpha tocopheryl polyethylene glycol-1000 succinate, the pegylated form of d-alpha-tocopherol known Vitamin E TPGS, is included herein as an ester of alpha-tocopherol.

### EXAMPLE

The present invention has proven beneficial to multiple sclerosis patients having chronic symptoms of muscle weakness, lack of coordination, and uncontrolled tremors. An MS patient orally ingested a 3 teaspoon dose of a tocopherol blend each day for six months. Each 3 teaspoon dose contained 6.0 grams total tocopherols (alpha-, beta-, gamma-, delta-) of which 3.6 grams was gamma-tocopherol. An improvement in symptoms was seen in one week. A significant improvement of all three symptoms was attained by six weeks. The patient reported a marked deterioration in symptoms when the high dose gamma-T therapy was interrupted for a few days. Thus, it was concluded that the high dose gamma-T caused a reduction in the rate of demyelination.

1. A method for treating multiple sclerosis, said method comprising: administering a compound selected from the group consisting of gamma-tocopherol, an ester of gamma-tocopherol, gamma-tocotrienol, an ester of gamma-tocotrienol, and a combination thereof in a therapeutically effective dosage for decreasing the rate of demyelination of the central nervous system of a patient having multiple sclerosis.

2. The method according to claim 1 wherein said ester of gamma-tocopherol and said ester of gamma-tocotrienol is each selected from the group consisting of succinate, polyethylene glycol succinate, acetate, nicotinate, and phosphate.

3. The method according to claim 1 wherein said compound is gamma-tocopherol, an ester of gamma-tocopherol, or a combination thereof.

4. The method according to claim 1 wherein said therapeutically effective dosage includes an amount of from about 800 mg to about 10 grams of gamma-tocopherol, an ester of gamma-tocopherol, or a combination thereof per day.

5. The method according to claim 4 wherein said dosage of gamma-tocopherol, an ester of gamma-tocopherol, or a combination thereof is between from greater than about 1 gram to about 7.5 grams per day.

6. The method according to claim 1 wherein said therapeutically effective dosage includes an amount of from about 50 mg to about 5 grams of gamma-tocotrienol, an ester of gamma-tocotrienol, or a combination thereof per day.

7. The method according to claim 6 wherein said dosage of gamma-tocotrienol, an ester of gamma-tocotrienol, or a combination thereof is between about 100 mg to about 2 grams per day.

8. The method according to claim 1 wherein said administering is conducted under a chronic dosing regimen for a prolonged period of time.

9. The method according to claim 1 wherein said administering is conducted under an acute dosing regimen in response to an acute exacerbation of multiple sclerosis.

10. The method according to claim 1 wherein said method is conducted in conjunction with a multiple sclerosis drug therapy.

11. A medicament useful for reducing the rate of demyelination in multiple sclerosis, each dose of said medicament comprising: a therapeutically effective amount of a demyelinating agent selected from the group consisting of gamma-tocopherol, an ester of gamma-tocopherol, gamma-tocotrienol, an ester of gamma-tocotrienol, and a combination thereof.

12. The medicament according to claim 11 wherein each said dose includes from about 800 mg to about 10 grams of gamma-tocopherol, an ester of gamma-tocopherol, or a combination thereof.

13. The medicament according to claim 12 wherein each said dose includes from about 1 gram to about 5 grams of gamma-tocopherol, an ester of gamma-tocopherol, or a combination thereof.

14. The medicament according to claim 11 wherein each said dose includes from about 50 mg to about 5 grams of gamma-tocotrienol, an ester of gamma-tocotrienol, or a combination thereof.

15. The medicament according to claim 14 wherein each said dose includes from about 100 mg to about 2 grams of gamma-tocotrienol, an ester of gamma-tocotrienol, or a combination thereof.

16. The medicament according to claim 11 further comprising a concentration of coenzyme Q10 wherein the weight of said demyelinating agent is at least two times greater than the weight of said coenzyme Q10.

17. The medicament according to claim 16 further comprising other nutrients selected from the group consisting of omega-3 fatty acids, omega-6 fatty acids, alpha-lipoic acid, astaxanthin, alpha-tocopherol, beta-tocopherol, delta-tocopherol, alpha-tocotrienol, beta-tocotrienol, delta-tocotrienol, genistein, daidzein, glycitein, equol, formononetin, biochanin and a combination thereof.

18. The medicament according to claim 11 wherein said ester of gamma-tocopherol and said ester of gamma-tocotrienol is each selected from the group consisting of succinate, polyethylene glycol succinate, acetate, nicotinate, and phosphate.

19. An oral dosage form of the medicament according to claim 11.

20. An injectable dosage form of the medicament according to claim 11.

21. A composition useful for treating multiple sclerosis, said composition comprising: an admixture including a drug selected from the group consisting of immunomodulators, corticosteroids, and immunosuppressors; and a demyelinating agent selected from the group consisting of gamma-tocopherol, an ester of gamma-tocopherol, gamma-tocotrienol, an ester of gamma-tocotrienol, and a combination thereof, wherein said demyelinating agent is present in said admixture at a therapeutically effective concentration for reducing the rate of demyelination.
